(19) United States
(12) Patent Application Publication
(10) Pub. No.: US 2024/0383871 A1
(43) Pub. Date: Nov. 21, 2024

(54) COMPOUND USEFUL FOR PET-IMAGING OF BRUTON’S TYROSINE KINASE

(71) Applicant: Bristol-Myers Squibb Company, Princeton, NJ (US)

(72) Inventors: David J. Donnelly, Doylestown, PA (US); Alban J. Allentoff, Flemington, NJ (US); Michael Arthur Wallace, Ringoes, NJ (US); Samuel J. Bonacorsi, Flemington, NJ (US); Scot Watterson, Pennington, NJ (US); Joseph A. Tino, Lawrenceville, NJ (US)

(73) Assignee: Bristol-Myers Squibb Company, Princeton, NJ (US)

(21) Appl. No.: 18/687,356

(22) PCT Filed: Aug. 29, 2022
(86) PCT No.: PCT/US2022/075556

§ 371 Date: Feb. 28, 2024

Related U.S. Application Data
(60) Provisional application No. 63/238,255, filed on Aug. 30, 2021.

(51) Int. Cl. C07D 401/04; A61K 51/04; C07B 59/00

(52) U.S. Cl. CPC C07D 401/04; A61K 51/0455; C07B 59/002

(57) ABSTRACT

Disclosed is a compound of Formula (Ib): The compound of Formula (Ib) is useful for positron emission tomography (PET) imaging of Bruton's Tyrosine Kinase (BTK) in mammals. Also disclosed are methods of using the compound as a labeling and diagnostic imaging agent of Bruton's Tyrosine Kinase (BTK), and methods of preparing Compound (Ib).

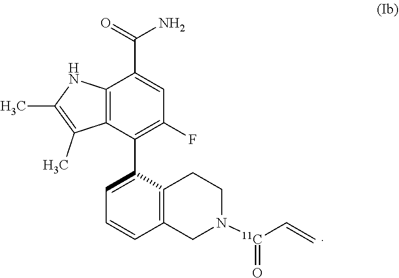

### CROSS-REFERENCE TO RELATED APPLICATIONS

This application claims the benefit of U.S. Provisional Application Ser. No. 63/238,255, filed Aug. 30, 2021, which is incorporated herein in its entirety.

### FIELD OF THE INVENTION

The invention generally relates to a radiolabeled Bruton's tyrosine kinase (BTK) compound and its use in labeling and diagnostic imaging of BTK in mammals.

### BACKGROUND OF THE INVENTION

Positron emission tomography (PET) is a non-invasive imaging technique that can provide functional information about biological processes in living subjects. The ability to image and monitor in vivo molecular events, is useful to gain insight into biochemical and physiological processes in living organisms. This in turn is essential for the development of novel approaches for the treatment of diseases, early detection of disease and for the design of new drugs. PET relies on the design and synthesis of molecules labeled with positron-emitting radioisotope. These molecules are known as radiotracers or radioligands. For PET imaging, the most commonly used positron emitting (PET) radionuclides are: ¹⁸F, ¹¹C, ¹⁵O and ¹³N, all of which are cyclotron produced, and have half-lives of 110, 20, 2 and 10 minutes, respectively. After being radiolabeled with a positron emitting radionuclide, these PET radioligands are administered to mammals, typically by intravenous (i.v.) injection. Once inside the body, the radioligand decays and emits a positron that travels a small distance until it combines with an electron. An event known as an annihilation event then occurs, which generates two collinear photons with energies of 511 keV each. Using a PET imaging scanner, which is capable of detecting the gamma radiation emitted from the radioligand, planar and tomographic images reveal the distribution of the radiotracer as a function of time. PET radioligands provide useful in-vivo information relating to target engagement and dose dependent receptor occupancy for human receptors.

Multiple sclerosis is a chronic inflammatory demyelinating and neurodegenerative disease of the central nervous system. It is characterized by either relapses or accumulation of several neurological symptoms and pathologically by areas of mononuclear cell infiltrates, demyelination with incomplete remyelination and axonal loss throughout the brain and spinal cord. The activity and interactions of B cells, T cells and myeloid cells are involved in the immunopathological features of multiple sclerosis. Activated B cells can exert effector functions through antigen presentation and cytokine production. Macrophages and microglia are abundant in multiple sclerosis and contribute to tissue damage and impair tissue repair. Bruton's tyrosine kinase (BTK) is a member of the tyrosine-protein kinase family of kinases that transmit signals through a variety of B cell receptors and myeloid cells. Recently, inhibitors of BTK have been investigated as potential treatments for multiple sclerosis (Nature Biotechnology Vol. 39(1), 3-5 (2021)).

U.S. Pat. No. 9,802,915 B2 discloses compounds useful as inhibitors of BTK for use in treating BTK-dependent or BTK-mediated conditions or diseases such as multiple sclerosis.

Use of a specific PET radioligand having high affinity for BTK in conjunction with supporting imaging technology provides a method for clinical evolution around both target engagement and dose/occupancy relationships of BTK inhibitors in the human brain or in other organs such as the spleen kidneys, liver, or heart that express this target.

The preparation of a PET compound containing a short-lived radionuclide requires that the PET compound is synthesized, purified, formulated into a pharmaceutical dose, and administered to a patient within a short period of time in order to minimize loss through radioactive decay of the radionuclide label. Preparation times of roughly 2-3 physical half-lives of the employed radionuclide are desired. Longer preparation times for the PET compound leads to increased loss of the radionuclide label prior to administration to the patient, and may lead to the need to use higher dosages of the PET compound.

The selection of the PET molecule is determined in part by the ability to incorporate the short-lived PET radionuclide into a location in the PET molecule at the last stages of the synthesis process. After production of the PET radionuclide in a cyclotron, the radioactive molecule containing the PET radionuclide is isolated and then quickly incorporated into the PET molecule precursor with a minimal number of synthetic steps requiring a minimal amount of time. Incorporating the PET radionuclide in the last stages of the synthesis also allows the synthesis chemist to work with non-radioactive material in the earlier stages of the synthesis, and limits the handling of radioactive material until the last synthesis stages. The use of special equipment and protective procedures for working with radioactive material is not required until the last synthesis stages.

Carbon-11 bas a 20 minute half-life. Labeling with carbon-11 requires quick and efficient methods to maximize radiotracer yields (Långström et al., Journal of Labelled Compounds and Radiopharmaceuticals 2007, 50: 794-810; Dahl et al., Clin Transl Imaging (2017) 5:275-289).

There remains a need for a radiolabeled compound useful for PET imaging of Bruton's tyrosine kinase.

Further, there is a need for a radiolabeled compound useful for PET imaging of Bruton's tyrosine kinase that can be synthesized, purified, formulated and administered to a patient within a timeframe of three or less than three physical half-lives of the employed radionuclide.

Furthermore, there is a need for radiolabeled compound useful for PET imaging that has a high affinity for BTK.

Still furthermore, there still remains a need for a radiolabeled compound having a high affinity for BTK with the ability to cross the brain-blood barrier for PET imaging of Bruton's tyrosine kinase in the brain.

### SUMMARY OF THE INVENTION

The present invention fills the foregoing need by providing a ¹¹C radiolabeled BTK inhibitor compound useful for the exploratory and diagnostic imaging applications, both in-vitro and in-vivo, and for competition studies using radiolabeled and unlabeled BTK inhibitors.

Applicants have found a ¹¹C radiolabeled BTK inhibitor compound useful for PET imaging of Bruton's tyrosine kinase.

Applicants have found a ¹¹C radiolabeled BTK inhibitor compound useful for PET imaging of Bruton's tyrosine kinase that can be synthesized, purified, formulated and administered to a patient within a timeframe of three or less than three physical half-lives of the employed radionuclide.

Applicants have found a ¹¹C radiolabeled BTK inhibitor compound useful for PET imaging of Bruton's tyrosine kinase that has a high affinity for BTK.

Applicants have found a ¹¹C radiolabeled BTK inhibitor compound useful for PET imaging of Bruton's tyrosine kinase with the ability to cross the brain-blood barrier for PET imaging of Bruton's tyrosine kinase in the brain.

The present invention also provides pharmaceutical compositions comprising the ¹¹C radiolabeled BTK inhibitor compound and a pharmaceutically-acceptable carrier.

The present invention also provides a method of imaging BTK expression in mammalian tissues using the ¹¹C radiolabeled BTK inhibitor compound.

The present invention also provides methods for preparing the ¹¹C radiolabeled BTK inhibitor compound.

These and other features of the invention will be set forth in expanded form as the disclosure continues.

### BRIEF DESCRIPTION OF THE DRAWINGS

FIG. 1: Representative semi-preparative HPLC chromatogram of the purification of [¹¹C]-(R)-4-(2-acryloyl-1,2,3,4-tetrahydroisoquinolin-5-yl)-5-fluoro-2,3-dimethyl-1H-indole-7-carboxamide using method A. [¹¹C]-(R)-4-(2-acryloyl-1,2,3,4-tetrahydroisoquinolin-5-yl)-5-fluoro-2,3-dimethyl-1H-indole-7-carboxamide was isolated between 8.5-9.5 minutes post injection onto the HPLC.

FIG. 2: Co-injection of [¹¹C]-(R)-4-(2-acryloyl-1,2,3,4-tetrahydroisoquinolin-5-yl)-5-fluoro-2,3-dimethyl-1H-indole-7-carboxamide and (R)-4-(2-acryloyl-1,2,3,4-tetrahydroisoquinolin-5-yl)-5-fluoro-2,3-dimethyl-1H-indole-7-carboxamide, non-radioactive reference standard using analytical reverse phase HPLC. Peaks co-eluted within 0.1 minutes using this HPLC analytical method.

FIG. 3: Representative semi-preparative HPLC chromatogram of purification of [¹¹C]-(R)-4-(2-acryloyl-1,2,3,4-tetrahydroisoquinolin-5-yl)-5-fluoro-2,3-dimethyl-1H-indole-7-carboxamide using method A. [¹¹C]-(R)-4-(2-acryloyl-1,2,3,4-tetrahydroisoquinolin-5-yl)-5-fluoro-2,3-dimethyl-1H-indole-7-carboxamide was isolated between 9-10.5 minutes post injection onto the HPLC.

FIG. 4: Standard uptake values (SUV) of [¹¹C]-(R)-4-(2-acryloyl-1,2,3,4-tetrahydroisoquinolin-5-yl)-5-fluoro-2,3-dimethyl-1H-indole-7-carboxamide PET signal showing retention of ligand within the EAE lesion region of the mouse brain in 2 EAE induced mice. The left bars indicate SUV values within the lesion region of the mouse brain 50 minutes after ligand administration. The right bars indicate follow-up SUV values within the lesion region of the mouse brain 50 minutes after ligand administration within the same animals. These follow-up images were acquired 24 hours later after a second administration of the PET ligand. Each set of bars represents data from an individual mouse.

FIG. 5: Representative PET images showing brain accumulation of [¹¹C]-(R)-4-(2-acryloyl-1,2,3,4-tetrahydroisoquinolin-5-yl)-5-fluoro-2,3-dimethyl-1H-indole-7-carboxamide (SUV from 20-50 min following PET ligand administration) (A) Baseline in WT mouse (B) Baseline in EAE induced mouse. The circle indicates the brain area in each animal and contrast ratio was calculated from the SUV measured in lesion region compared to the SUV in the area outside the lesion region of the brain, this ratio was 4.7:1.

FIG. 6: Representative bar graphs from quantification of the PET images in EAE induced mouse brains. Two regions of interest were defined within this study, the lesion area and an area of the brain outside of lesions, showing brain accumulation of [¹¹C]-(R)-4-(2-acryloyl-1,2,3,4-tetrahydroisoquinolin-5-yl)-5-fluoro-2,3-dimethyl-1H-indole-7-carboxamide (SUV from 50 minutes following PET ligand administration). The left bar represents the lesion area within the brain. The next bar represents the lesion area during the dosing panel (either 5 or 0.5 mg/kg BTK inhibitor. The right bar represents a representative ROI within the mouse brain, outside of the lesion region at baseline. The bar represents a representative ROI within the mouse brain, outside of the lesion region during dosing of BTK inhibitor.

FIG. 7: Representative rhesus monkey image and TAC of [¹¹C]-(R)-4-(2-acryloyl-1,2,3,4-tetrahydroisoquinolin-5-yl)-5-fluoro-2,3-dimethyl-1H-indole-7-carboxamide in a healthy NHP at baseline. The circle area indicates the brain area of the NHP within the PET scan image.

The present disclosure is based, in part, on the appreciation that the radiolabeled Burton tyrosine kinase (hereinafter “BTK”) inhibitor is useful in the detection and/or quantification and/or imaging of BTK and/or BTK expression and/or affinity of a compound for occupying BTK receptors, in tissue of a mammalian species. It has been found that the radiolabeled BTK inhibitor, when administered to a mammalian species, builds up at or occupies BTK and can be detected through imaging techniques, thereby providing valuable diagnostic markers for presence of BTK in tissues, affinity of a compound for occupying BTK, and clinical evaluation and dose selection of BTK inhibitors. In addition, the radiolabeled BTK inhibitor disclosed herein can be used as a research tool to study the interaction of unlabeled BTK inhibitors with BTK in vivo via competition between the unlabeled drug and the radiolabeled drug for binding to the receptor. These types of studies are useful in determining the relationship between BTK receptor occupancy and dose of unlabeled BTK inhibitors, as well as for studying the duration of blockade of the binding site by various doses of unlabeled BTK inhibitors.

As a clinical tool, the radiolabeled BTK inhibitor can be used to help define clinically efficacious doses of BTK inhibitors. In animal experiments, the radiolabeled BTK inhibitor can be used to provide information that is useful for choosing between potential drug candidates for selection for clinical development. The radiolabeled BTK inhibitor can also be used to study the regional distribution and concentration of BTK in living lung tissue and other tissue, such as kidney, heart, liver and skin, of humans and animals and in tissue samples. They can be used to study disease or pharmacologically related changes in BTK concentrations.

### DETAILED DESCRIPTION

WO 2016/065226 discloses the non-PET labeled compound 4-(2-acryloy 1-1, 2,3,4-tetrahydroisoquinolin-5-yl)-5-fluoro-2,3-dimethyl-IH-indole-7-carboxamide in Example 95 as a racemic mixture: and the two individual enantiomers in Examples 153 and 154. Example 154 is non-PET labeled (R)-4-(2-acryloyl-1,2,3,4-tetrahydroisoquinolin-5-yl)-5-fluoro-2,3-dimethyl-1H-indole-7-carboxamide and is referred to herein as “Compound (Ia). Compound (Ia) has the structure:

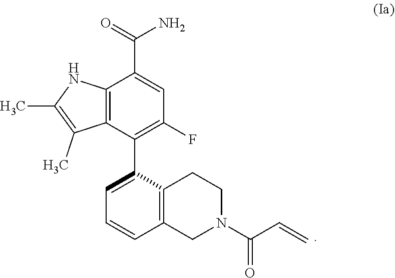

WO 2016/065226 discloses a BTK IC₅₀ inhibition value of 0.10 nM for Example 154.

The first aspect of the present invention provides a compound of Formula (Ib) having the structure:

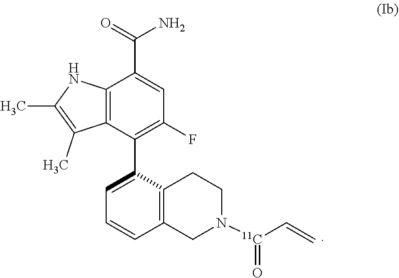

The compound of Formula (Ib) is a ¹¹C radiolabeled BTK inhibitor compound and is also referred to herein as the ““ Compound (Ib)”. The chemical name for Compound (Ib) is [¹¹C]-(R)-4-(2-acryloyl-1,2,3,4-tetrahydroisoquinolin-5-yl)-5-fluoro-2,3-dimethyl-1H-indole-7-carboxamide.

The second aspect of the invention provides a pharmaceutical composition comprising the compound of Formula (Ib) and a pharmaceutically acceptable carrier.

The third aspect of the invention provides methods of using the compound of Formula (Ib) for imaging, screening, and/or monitoring.

The fourth aspect of the invention provides methods of preparing the compound of Formula (Ib).

As used herein, the term “Compound (I)” refers to a mixture of Compound (Ia) and Compound (Ib). Compound (I) includes mixtures having from zero mole % Compound (Ia) and 100 mole % Compound (Ib) to a mixture having 100 mole % Compound (Ia) and zero mole % Compound (Ib). For example, Compound (I) includes mixtures of: 1 mole % Compound (Ia) and 99 mole % Compound (Ib); 5 mole % Compound (Ia) and 95 mole % Compound (Ib); 10 mole % Compound (Ia) and 90mole % Compound (Ib); 20 mole % Compound (Ia) and 80 mole % Compound (Ib); 30mole % Compound (Ia) and 70 mole % Compound (Ib); 40 mole % Compound (Ia) and 60 mole % Compound (Ib); 50 mole % Compound (Ia) and 50 mole % Compound (Ib); 70 mole % Compound (Ia) and 30 mole % Compound (Ib); 80 mole % Compound (Ia) and 20 mole % Compound (Ib); 90 mole % Compound (Ia) and 10 mole % Compound (Ib); 95 mole % Compound (Ia) and 5 mole % Compound (Ib); and 100 mole % Compound (Ia) and 0 mole % Compound (Ib).

As used herein, the term “molar activity of Compound (I)” refers to the measured radioactivity per mole of Compound (I). Suitable units for molar activity include gigabecquerel/mole (GBq/mol).

As used herein, the term “specific activity of Compound (I)” refers to the measured radioactivity per gram of Compound(I). Suitable units for molar activity include gigabecquerel/milligram (GBq/mg).

### Pharmaceutical Composition

The second aspect of the invention provides a pharmaceutical composition comprising the compound of Formula (Ib) and a pharmaceutically acceptable carrier. Examples of suitable pharmaceutically acceptable carriers include sterile saline solution, ethanol, and sodium ascorbate.

One embodiment provides the pharmaceutical composition comprising the compound of Formula (Ib) and a pharmaceutically acceptable carrier, wherein the dosage of the compound of Formula (Ib) is in the range of from 0.2 μg to 100 μg. Included in this embodiment is a pharmaceutical composition comprising the compound of Formula (Ib) wherein the dosage is in the range of from 0.5 μg to 90 μg. Also included in this embodiment is a pharmaceutical composition comprising the compound of Formula (Ib) wherein the dosage is in the range of from 1 μg to 75 μg.

One embodiment provides the pharmaceutical composition comprising 5 to 25 millicuries of the compound of Formula (Ib) and a pharmaceutically acceptable carrier.

One embodiment provides the pharmaceutical composition comprising 5 to 22 millicuries of the compound of Formula (Ib) and a pharmaceutically acceptable carrier.

One embodiment provides the pharmaceutical composition comprising 5 to 20 millicuries of the compound of Formula (Ib) and a pharmaceutically acceptable carrier.

One embodiment provides the pharmaceutical composition comprising 7 to 20 millicuries of the compound of Formula (Ib) and a pharmaceutically acceptable carrier.

One embodiment provides the pharmaceutical composition comprising 10 to 20 millicuries of the compound of Formula (Ib) and a pharmaceutically acceptable carrier.

One embodiment provides the pharmaceutical composition comprising 7 to 15 millicuries of the compound of Formula (Ib) and a pharmaceutically acceptable carrier.

One embodiment provides the pharmaceutical composition comprising 10 to 15 millicuries of the compound of Formula (Ib) and a pharmaceutically acceptable carrier.

One embodiment provides the pharmaceutical composition comprising 0.2 μg of Compound (Ib) and a pharmaceutically acceptable carrier. Included in this embodiment is a pharmaceutical composition comprising from 0.2 μg to 100 μg of Compound (Ib) and a pharmaceutically acceptable carrier.

One embodiment provides the pharmaceutical composition comprising 0.5 μg of Compound (Ib) and a pharmaceutically acceptable carrier. Included in this embodiment is a pharmaceutical composition comprising from 0.5 μg to 100 μg of Compound (Ib) and a pharmaceutically acceptable carrier.

One embodiment provides the pharmaceutical composition comprising 1 μg of Compound (Ib) and a pharmaceutically acceptable carrier. Included in this embodiment is a pharmaceutical composition comprising from 1 μg to 100 μg of Compound (Ib) and a pharmaceutically acceptable carrier.

One embodiment provides the pharmaceutical composition comprising 2 μg of Compound (Ib) and a pharmaceutically acceptable carrier. Included in this embodiment is a pharmaceutical composition comprising from 2 μg to 100 μg of Compound (Ib) and a pharmaceutically acceptable carrier.

One embodiment provides the pharmaceutical composition comprising 5 μg of Compound (Ib) and a pharmaceutically acceptable carrier. Included in this embodiment is a pharmaceutical composition comprising from 5 μg to 100 μg of Compound (Ib) and a pharmaceutically acceptable carrier.

One embodiment provides the pharmaceutical composition comprising 10 μg of Compound (Ib) and a pharmaceutically acceptable carrier. Included in this embodiment is a pharmaceutical composition comprising from 10 μg to 100 μg of Compound (Ib) and a pharmaceutically acceptable carrier.

One embodiment provides the pharmaceutical composition comprising 15 μg of Compound (Ib) and a pharmaceutically acceptable carrier. Included in this embodiment is a pharmaceutical composition comprising from 15 μg to 100 μg of Compound (Ib) and a pharmaceutically acceptable carrier.

One embodiment provides the pharmaceutical composition comprising 20 μg of Compound (Ib) and a pharmaceutically acceptable carrier. Included in this embodiment is a pharmaceutical composition comprising from 20 μg to 100 μg of Compound (Ib) and a pharmaceutically acceptable carrier.

One embodiment provides the pharmaceutical composition comprising 30 μg of Compound (Ib) and a pharmaceutically acceptable carrier. Included in this embodiment is a pharmaceutical composition comprising from 30 μg to 100 μg of Compound (Ib) and a pharmaceutically acceptable carrier.

One embodiment provides the pharmaceutical composition comprising 40 μg of Compound (Ib) and a pharmaceutically acceptable carrier. Included in this embodiment is a pharmaceutical composition comprising from 40 μg to 100 μg of Compound (Ib) and a pharmaceutically acceptable carrier.

One embodiment provides the pharmaceutical composition comprising 50 μg of Compound (Ib) and a pharmaceutically acceptable carrier. Included in this embodiment is a pharmaceutical composition comprising from 50 μg to 100 μg of Compound (Ib) and a pharmaceutically acceptable carrier.

According to one embodiment of the present invention, pharmaceutical and diagnostic compositions are provided. Such pharmaceutical or diagnostic composition comprises a compound of Formula (Ib): and a pharmaceutically acceptable carrier therefor. In one embodiment, the compound of Formula (Ib) is present in a therapeutically effective amount and diagnostically effect amount in the pharmaceutical and diagnostic compositions, respectively.

### METHODS OF USE

The present disclosure is based, in part, on the appreciation that a radiolabeled Burton tyrosine kinase (hereinafter “BTK”) inhibitor is useful in the detection and/or quantification and/or imaging of BTK and/or BTK expression and/or affinity of a compound for occupying BTK receptors, in tissue of a mammalian species. It has been found that a radiolabeled BTK inhibitor, when administered to a mammalian species, build up at or occupy BTK and can be detected through imaging techniques, thereby providing valuable diagnostic markers for presence of BTK in tissues, affinity of a compound for occupying BTK, and clinical evaluation and dose selection of BTK inhibitors. In addition, the radiolabeled BTK inhibitor disclosed herein can be used as a research tool to study the interaction of unlabeled BTK inhibitors with BTK in vivo via competition between the unlabeled drug and the radiolabeled drug for binding to the receptor. These types of studies are useful in determining the relationship between BTK receptor occupancy and dose of unlabeled BTK inhibitors, as well as for studying the duration of occupancy of the binding site by various doses of unlabeled BTK inhibitors.

As a clinical tool, the radiolabeled BTK inhibitor, the compound of Formula (Ib), can be used to help define clinically efficacious doses of BTK inhibitors. In animal experiments, the radiolabeled BTK inhibitor can be used to provide information that is useful for choosing between potential drug candidates for selection for clinical development. The radiolabeled BTK inhibitor can also be used to study the regional distribution and concentration of BTK in living brain tissue, lung tissue and other tissue, such as kidney, heart, liver and skin, of humans and animals and in tissue samples. They can be used to study disease or pharmacologically related changes in BTK concentrations.

One embodiment provides a method of in vivo imaging of mammalian tissues of known BTK expression comprising the steps of:
(a) administering the radiolabeled compound of Formula (Ib) to a mammalian species; and
(b) imaging in vivo the distribution of the radiolabeled compound by positron emission tomography (PET) scanning.
   Included in this embodiment is a method of in vivo imaging of mammalian living brain tissue of known BTK expression. Additionally, included in this embodiment is a method of in vivo imaging of human living brain tissue of known BTK expression.

In another embodiment, the present invention provides a method for screening a non-radiolabeled compound to determine its affinity for occupying the binding sites of BTK in mammalian tissue comprising the steps of:
(a) administering the radiolabeled compound of Formula (Ib) to a mammalian species;
(b) imaging in vivo tissues of known BTK expression by positron emission tomography (PET) to determine a baseline uptake of the radiolabeled compound;
(c) administering the non-radiolabeled compound to the mammalian species;
(d) administering a second dose of the radiolabeled compound of Formula (Ib) to the mammalian species;
(e) imaging in vivo the distribution of the radiolabeled compound of Formula (Ib) in tissues that express BTK;
(f) comparing the signal from PET scan data at the baseline within the tissue that expresses BTK to PET scan data retrieved after administering the non-radiolabeled compound within the tissue that expresses BTK receptors.
   Included in this embodiment is a method for screening a non-radiolabeled compound to determine its affinity for occupying the binding sites of BTK in mammalian tissue wherein the mammalian tissue is human tissue. Included in this embodiment is a method for screening a non-radiolabeled compound to determine its affinity for occupying the binding sites of BTK in mammalian tissue wherein the mammalian tissue is human brain tissue.

In another embodiment, the present invention provides a method for monitoring the treatment of a mammalian patient who is being treated with an BTK inhibitor comprising the steps of:
(a) administering to the patient the radiolabeled compound of Formula (Ib);
(b) obtaining an image of tissues in the patient that express BTK by positron emission tomography (PET); and
(c) detecting to what degree the radiolabeled compound occupies the binding site of the BTK.

In another embodiment, the present invention provides a method for monitoring the treatment of a mammalian patient who is being treated with an BTK inhibitor comprising the steps of:
(a) administering to the patient the radiolabeled compound of Formula (Ib);
(b) obtaining an image of brain tissue in the patient that express BTK by positron emission tomography (PET); and
(c) detecting to what degree the radiolabeled compound occupies the binding site of the BTK.

In another embodiment, the present invention provides a method for tissue imaging comprising the steps of contacting a tissue that contains BTK with the radiolabeled compound of Formula (Ib) and detecting the radiolabeled compound using positron emission tomography (PET) imaging. In such a method, the radiolabeled compound can be detected either in vitro or in vivo.

In another embodiment, the present invention provides a method for diagnosing the presence of multiple sclerosis in a mammalian species, comprising the steps of
(a) administering to a mammalian species in need thereof the radiolabeled compound of Formula (Ib), which binds to the BTK associated with the presence of the multiple sclerosis disease; and
(b) obtaining a radio-image of at least a portion of the mammalian species to detect the presence or absence of the radiolabeled compound; wherein the presence and location of the radiolabeled compound above background is indicative of the presence or absence of the disease.

In another embodiment, the present invention provides a method for diagnosing the presence of multiple sclerosis in a mammalian species, comprising the steps of
(a) administering to a mammalian species in need thereof the radiolabeled compound of Formula (Ib), which binds to the BTK associated with the presence of the multiple sclerosis disease; and
(b) obtaining a PET scan or autoradiogram of brain tissue of the mammalian species to detect the presence or absence of the radiolabeled compound: wherein the presence and location of the radiolabeled compound above background is indicative of the presence or absence of the disease.

In another embodiment, the present invention provides a method for diagnosing the presence of multiple sclerosis, in a mammalian species, comprising the steps of
(a) administering to a mammalian species in need thereof the radiolabeled compound of Formula (Ib), which binds to BTK associated with the presence of the multiple sclerosis;
(b) detecting radioactive emission of the radiolabeled compound for the mammalian species;
(c) comparing the radioactive emission from the radiolabeled compound for the mammalian species with standard values; and
(d) finding any significant deviation between the radioactive emission detected for the mammalian species as compared with standard values, and attributing the deviation to the multiple sclerosis.
   Included in this embodiment is a method for diagnosing the presence of multiple sclerosis in which the radioactive emission of the radiolabeled compound for the mammalian species in step (b) is detected in the brain tissue of the mammalian species.

In another embodiment, the present invention provides a method for quantifying diseased cells or tissues in a mammalian species, comprising the steps of
(a) administering to a mammalian species having diseased cells or tissues the radiolabeled compound of Formula (Ib), which binds to BTK located within the diseased cells or tissues; and
(b) detecting radioactive emissions of the radiolabeled compound in the diseased cells or tissues, wherein the level and distribution of the radioactive emissions in the diseased cells or tissues is of a quantitative measure of the diseased cells or tissues.
   Included in this embodiment is a method for quantifying diseased cells or tissues in a mammalian species wherein the diseased cells or tissues are diseased brain cells or brain tissues.

The compound of Formula (Ib) is a radiolabeled BTK inhibitor which is useful as a positron emitting molecule having BTK affinity. The term “radiolabeled BTK inhibitor” as used herein refers to a compound of Formula (Ib).

In another embodiment, the present disclosure provides a diagnostic composition for imaging BTK which includes a radiolabeled BTK inhibitor, i.e., a compound of Formula (Ib), and a pharmaceutically acceptable carrier therefor. In still another embodiment, the present disclosure provides a pharmaceutical composition which includes a radiolabeled BTK inhibitor, i.e., a compound of Formula (Ib), and a pharmaceutically acceptable carrier therefor. In yet another embodiment, the present disclosure provides a method of autoradiography of mammalian tissues of known BTK expression, which includes the steps of administering a radiolabeled BTK inhibitor to a mammalian species, obtaining an image of the tissues by positron emission tomography, and detecting the radiolabeled compound in the tissues to determine BTK inhibitor target engagement and BTK inhibitor receptor occupancy of said tissues.

Radiolabeled BTK inhibitors, when labeled with the appropriate radionuclide, are potentially useful for a variety of in vitro and/or in vivo imaging applications, including diagnostic imaging, basic research, and radiotherapeutic applications. Specific examples of possible diagnostic imaging and radiotherapeutic applications include determining the location of, the relative activity of and/or quantification of BTK; radioimmunoassay of BTK inhibitors; and autoradiography to determine the distribution of BTK in a mammal or an organ or tissue sample thereof.

In particular, the instant radiolabeled BTK inhibitor is useful for positron emission tomographic (PET) imaging of BTK in the brain, spleen, and other organs of living humans and experimental animals. This radiolabeled BTK inhibitor may be used as research tools to study the interaction of unlabeled BTK inhibitors with BTK in vivo via competition between the unlabeled drug and the radiolabeled compound for binding to the kinase binding site. These types of studies are useful for determining the relationship between BTK occupancy and dose of unlabeled BTK inhibitors, as well as for studying the duration of blockade of the receptor by various doses of the unlabeled BTK inhibitors. As a clinical tool, the radiolabeled BTK inhibitor may be used to help define a clinically efficacious dose of an BTK inhibitor. In animal experiments, the radiolabeled BTK inhibitor can be used to provide information that is useful for choosing between potential drug candidates for selection for clinical development. The radiolabeled BTK inhibitor may also be used to study the regional distribution and concentration of BTK in the human brain, spleen, and other organs of living experimental animals and in tissue samples, including healthy and diseased tissues, such as tumors. The radiolabeled BTK inhibitor may also be used to study disease or pharmacologically related changes in BTK concentrations.

For example, positron emission tomography (PET) tracers such as the present radiolabeled BTK inhibitor can be used with currently available PET technology to obtain the following information: relationship between level of receptor occupancy by candidate BTK inhibitor and clinical efficacy in patients; dose selection for clinical trials of BTK inhibitors prior to initiation of long term clinical studies; comparative potencies of structurally BTK inhibitors; investigating the influence of BTK inhibitors on in vivo transporter affinity and density during the treatment of clinical targets with BTK inhibitors; changes in the density and distribution of BTK during effective and ineffective treatment of multiple sclerosis and cancer.

The present radiolabeled BTK inhibitor has utility in imaging BTK for diagnostic imaging with respect to a variety of disorders associated with BTK.

For the use of the instant compound as exploratory or diagnostic imaging agent, the radiolabeled compound may be administered to mammals, preferably humans, in a pharmaceutical composition, either alone or, preferably, in combination with pharmaceutically acceptable carriers or diluents, optionally with known adjuvants, such as alum, in a pharmaceutical composition, according to standard pharmaceutical practice. Such compositions can be administered orally or parenterally, including the intravenous, intramuscular, intraperitoneal, subcutaneous, rectal and topical routes of administration. Preferably, administration is intravenous. The BTK PET radioligand is a radiotracer labeled with short-lived, positron emitting radionuclide and thus is generally administered via intravenous injection within less than one hour of their synthesis. This is necessary because of the short half-life of the radionuclide involved. When the present radiolabeled BTK PET radioligand is administered into a human subject, the amount required for imaging will normally be determined by the prescribing physician with the dosage generally varying according to the quantity of emission from the radionuclide. However, in most instances, an effective amount will be the amount of compound sufficient to produce emissions in the range of from about1-20 mCi. In one exemplary application, administration occurs in an amount between 0.5-20 mCi of total radioactivity injected into a mammal depending upon the subjects body weight. The upper limit is set by the dosimetry of the radiolabeled molecule in either rodent or non-human primate.

The following illustrative procedure may be utilized when performing PET imaging studies on patients in the clinic. The patient is premedicated with an unlabeled BTK inhibitor some time prior to the day of the experiment and is fasted for at least 12 hours allowing water intake ad libitum. A 20 G two-inch venous catheter is inserted into the contralateral ulnar vein for radiotracer administration. Administration of the PET tracer is often timed to coincide with time of maximum (Tₘₐₓ) or minimum (Tₘᵢₙ) of BTK inhibitors concentration in the blood.

The patient is positioned in the PET camera and a tracer dose of the radiolabeled BTK inhibitor such as Compound (Ib) (<20 mCi) is administered via i.v. catheter. Either arterial or venous blood samples are taken at appropriate time intervals throughout the PET scan in order to analyze and quantitate the fraction of unmetabolized PET tracer of [¹¹C] Compound (Ib) in plasma. Images are acquired for up to 120 min. Within ten minutes of the injection of radiotracer and at the end of the imaging session, 1 mL blood samples are obtained for determining the plasma concentration of any BTK inhibitors which may have been administered before the PET tracer.

Tomographic images are obtained through image reconstruction. For determining the distribution of radiotracer, regions of interest (ROIs) are drawn on the reconstructed image including, but not limited to, the brain, spleen, tumor, lungs, liver, heart, kidney, skin or other organs and tissue. Radiotracer uptakes over time in these regions are used to generate time activity curves (TAC) obtained in the absence of any intervention or in the presence of the unlabeled BTK inhibitors at the various dosing paradigms examined. Data are expressed as radioactivity per unit time per unit volume (μCi/cc/mCi injected dose). TAC data are processed with various methods well-known in the field to yield quantitative parameters, such as Binding Potential (BP) or Volume of Distribution (V_{T}), that are proportional to the density of unoccupied BTK within the region of interest. Inhibition of BTK inhibitors is then calculated based on the change of BP or VT by equilibrium analysis in the presence of BTK or BTK inhibitor at the various dosing paradigms as compared to the BP or V_{T} in the unmedicated state. Inhibition curves are generated by plotting the above data vs the dose (concentration) of BTK inhibitors. Inhibition of BTK inhibitor is then calculated based on the maximal reduction of PET radioligand's V_{T} or BP that can be achieved by a blocking drug at Eₘₐₓ, Tₘₐₓ or Tₘᵢₙ and the change of its non-specific volume of distribution (V_{ND}) and the BP in the presence of

BTK inhibitors at the various dosing paradigms as compared to the BP or V_{T} in the unmedicated state. The ID₅₀ values are obtained by curve fitting the dose-rate/inhibition curves.

The present disclosure is further directed to a method for the diagnostic imaging of BTK in a mammal in need thereof which includes the step of combining radiolabeled BTK inhibitors with a pharmaceutical carrier or excipient.

### Processes for Preparation of Compound of Formula (IB)

One embodiment provides a method of preparing [¹¹C]-(R)-4-(2-acryloyl-1,2,3,4-tetrahydroisoquinolin-5-yl)-5-fluoro-2,3-dimethyl-1H-indole-7-carboxamide, comprising the steps of:
(a) reacting [¹¹C]CO₂ and vinyl magnesium bromide to provide [¹¹C]-acrylic acid having the structure of Formula (II):

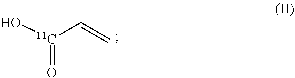

and
(b) reacting said [¹¹C]-acrylic acid and (R)-5-fluoro-2,3-dimethyl-4-(1,2,3,4-tetrahydroisoquinolin-5-yl)-1H-indole-7-carboxamide to provide [¹¹C]-(R)-4-(2-acryloyl-1,2,3,4-tetrahydroisoquinolin-5-yl)-5-fluoro-2,3-dimethyl-1H-indole-7-carboxamide.

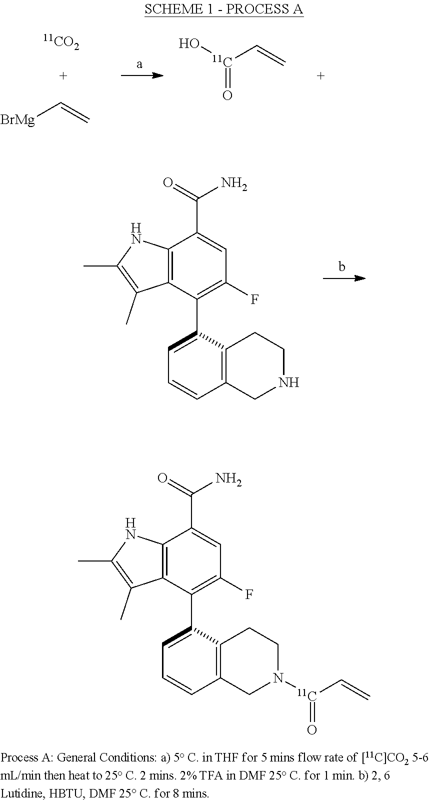

One embodiment provides a method of preparing [¹¹C]-(R)-4-(2-acryloyl-1,2,3,4-tetrahydroisoquinolin-5-yl)-5-fluoro-2,3-dimethyl-1H-indole-7-carboxamide, comprising the step of reacting [¹¹C]CO and vinyl iodide in the presence of (R)-5-fluoro-2,3-dimethyl-4-(1,2,3,4-tetrahydroisoquinolin-5-yl)-1H-indole-7-carboxamide to provide [¹¹C]-(R)-4-(2-acryloyl-1,2,3,4-tetrahydroisoquinolin-5-yl)-5-fluoro-2,3-dimethyl-1H-indole-7-carboxamide with vinyl magnesium bromide.

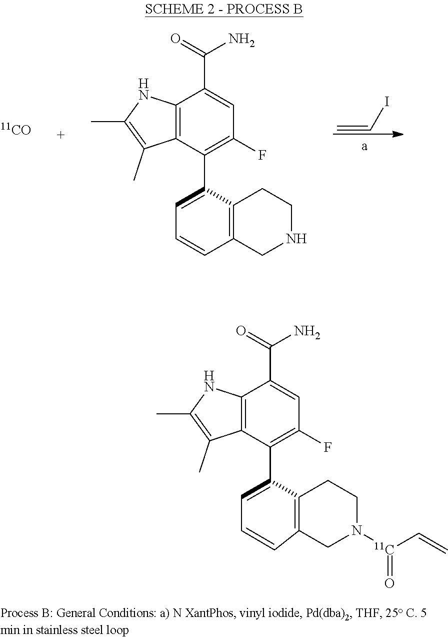

The present invention may be embodied in other specific forms without departing from the spirit or essential attributes thereof. This invention encompasses all combinations of the aspects and/or embodiments of the invention noted herein. It is understood that any and all embodiments of the present invention may be taken in conjunction with any other embodiment or embodiments to describe additional embodiments. It is also to be understood that each individual element of the embodiments is meant to be combined with any and all other elements from any embodiment to describe an additional embodiment.

The features and advantages of the invention may be more readily understood by those of ordinary skill in the art upon reading the following detailed description. It is to be appreciated that certain features of the invention that are, for clarity reasons, described above and below in the context of separate embodiments, may also be combined to form a single embodiment. Conversely, various features of the invention that are, for brevity reasons, described in the context of a single embodiment, may also be combined so as to form sub-combinations thereof. Embodiments identified herein as exemplary or preferred are intended to be illustrative and not limiting.

Unless otherwise stated, the following terms used in this application, including the specification and claims, have the definitions given below. It must be noted that, as used in the specification and the appended claims, the singular forms “a”, “an” and “the” include plural referents unless the context clearly dictates otherwise.

Unless otherwise indicated, conventional methods of mass spectroscopy, NMR, HPLC, protein chemistry, biochemistry, recombinant DNA techniques and pharmacology are employed. Furthermore, use of the term “including” as well as other forms, such as “include”, “includes”, and “included”, is not limiting. The section headings used herein are for organizational purposes only and are not to be construed as limiting the subject matter described.

The term “acceptable” with respect to a formulation, composition or ingredient, as used herein, means having no persistent detrimental effect on the general health of the subject being treated.

The term “modulate”, as used herein, means to interact with a target either directly or indirectly so as to alter the activity of the target, including, by way of example only, to enhance the activity of the target, to inhibit the activity of the target, to limit the activity of the target, or to extend the activity of the target.

Unless otherwise indicated, any atom with unsatisfied valences is assumed to have hydrogen atoms sufficient to satisfy the valences.

The definitions set forth herein take precedence over definitions set forth in any patent, patent application, and/or patent application publication incorporated herein by reference.

The phrase “pharmaceutically acceptable” is employed herein to refer to those compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

The compounds of Formula (Ib) can be provided as amorphous solids or crystalline solids. Lyophilization can be employed to provide the compounds of Formula (Ib) as a solid.

It should further be understood that solvates (e.g., hydrates) of the Compounds of Formula (Ib) are also within the scope of the present invention. The term “solvate” means a physical association of a compound of Formula (Ib) with one or more solvent molecules, whether organic or inorganic. This physical association includes hydrogen bonding. In certain instances the solvate will be capable of isolation, for example when one or more solvent molecules are incorporated in the crystal lattice of the crystalline solid. “Solvate” encompasses both solution-phase and isolable solvates. Exemplary solvates include hydrates, ethanolates, methanolates, isopropanolates, acetonitrile solvates, and ethyl acetate solvates. Methods of solvation are known in the art.

Various forms of prodrugs are well known in the art and are described in Rautio, J. et al., Nature Review Drug Discovery, 17, 559-587 (2018).

In addition, compounds of Formula (Ib), subsequent to their preparation, can be isolated and purified to obtain a composition containing an amount by weight equal to or greater than 99% of a compound of Formula (Ib) (“substantially pure”), which is then used or formulated as described herein. Such “substantially pure” compounds of Formula (Ib) are also contemplated herein as part of the present invention.

The terms “treat,” “treating,” and “treatment,” as used herein, refer to any type of intervention or process performed on, or administering an active agent to, the subject with the objective of reversing, alleviating, ameliorating, inhibiting, or slowing down or preventing the progression, development, severity or recurrence of a symptom, complication, condition or biochemical indicia associated with a disease. In contrast, “prophylaxis” or “prevention” refers to administration to a subject who does not have a disease to prevent the disease from occurring. “Treat,” “treating,” and “treatment” does not encompass prophylaxis or prevention.

“Therapeutically effective amount” is intended to include an amount of a compound of the present invention alone or an amount of the combination of compounds claimed or an amount of a compound of the present invention in combination with other active ingredients effective to decrease Helios protein levels, decrease Helios activity levels and/or inhibit Helios expression levels in the cells, or effective to treat or prevent viral infections and proliferative disorders, such as cancer.

As used herein, the term “cell” is meant to refer to a cell that is in vitro, ex vivo or in vivo. In some embodiments, an ex vivo cell can be part of a tissue sample excised from an organism such as a mammal. In some embodiments, an in vitro cell can be a cell in a cell culture. In some embodiments, an in vivo cell is a cell living in an organism such as a mammal.

The term “patient” includes human and other mammalian subjects that receive either therapeutic or prophylactic treatment.

The term “subject” includes any human or non-human animal. For example, the methods and compositions herein disclosed can be used to treat a subject having cancer. A non-human animal includes all vertebrates, e.g., mammals and non-mammals, including non-human primates, sheep, dogs, cows, chickens, amphibians, reptiles, etc. In one embodiment, the subject is a human subject.

The phrase “pharmaceutically acceptable carrier” as used herein means a pharmaceutically acceptable material, composition or vehicle, such as a liquid or solid filler, diluent, excipient, manufacturing aid (e.g., lubricant, talc magnesium, calcium or zinc stearate, or steric acid), or solvent encapsulating material, involved in carrying or transporting the subject compound from one organ, or portion of the body, to another organ, or portion of the body. Each carrier must be “acceptable” in the sense of being compatible with the other ingredients of the formulation, including, i.e., adjuvant, excipient or vehicle, such as diluents, preserving agents, fillers, flow regulating agents, disintegrating agents, wetting agents, emulsifying agents, suspending agents, sweetening agents, flavoring agents, perfuming agents, antibacterial agents, antifungal agents, lubricating agents and dispensing agents, depending on the nature of the mode of administration and dosage forms: and not injurious to the patient.

The term “pharmaceutical composition” means a composition comprising a compound of the invention in combination with at least one additional pharmaceutically acceptable carrier.

### Utility

The compound of Formula (I) is useful for the treatment of Crohn's disease, ulcerative colitis, asthma, atopic dermatitis, Graves' disease, rheumatoid arthritis, systemic lupus erythematosus (SLE), psoriasis, multiple sclerosis, Sjögren's syndrome, Alzheimer's disease, and Parkinson's disease.

In one embodiment, the present invention provides a combined preparation of a compound of Formula (I) and additional therapeutic agent(s) for simultaneous, separate or sequential use in the treatment and/or prophylaxis of multiple diseases or disorders associated with the activity of Bruton's tyrosine kinase. The combined preparation can be used to inhibit Bruton's tyrosine kinase.

### PHARMACEUTICAL COMPOSITIONS

The invention also provides pharmaceutically compositions which comprise a therapeutically effective amount of the compound of Formula (Ib), formulated together with one or more pharmaceutically acceptable carriers (additives) and/or diluents, and optionally, one or more additional therapeutic agents described above.

The compound of Formula (Ib) can be administered intravenously, subcutaneously, and/or intramuscularly via any pharmaceutically acceptable and suitable injectable form. Exemplary injectable forms include, but are not limited to, for example, sterile aqueous solutions comprising acceptable vehicles and solvents, such as, for example, water, Ringer's solution, and isotonic sodium chloride solution: sterile oil-in-water microemulsions: and aqueous or oleaginous suspensions.

Formulations for parenteral administration may be in the form of aqueous or non-aqueous isotonic sterile injection solutions or suspensions. These solutions and suspensions may be prepared using one or more of the carriers or diluents mentioned for use in the formulations for oral administration or by using other suitable dispersing or wetting agents and suspending agents. The compound may be dissolved in water, polyethylene glycol, propylene glycol, ethanol, corn oil, cottonseed oil, peanut oil, sesame oil, benzyl alcohol, sodium chloride, tragacanth gum, and/or various buffers. Other adjuvants and modes of administration are well and widely known in the pharmaceutical art. The active ingredient may also be administered by injection as a composition with suitable carriers including saline, dextrose, or water, or with cyclodextrin (i.e. Captisol), cosolvent solubilization (i.e. propylene glycol) or micellar solubilization (i.e. Tween 80).

The sterile injectable preparation may also be a sterile injectable solution or suspension in a non-toxic parenterally acceptable diluent or solvent, for example as a solution in 1,3-butanediol. Among the acceptable vehicles and solvents that may be employed are water, Ringer's solution, and isotonic sodium chloride solution. In addition, sterile, fixed oils are conventionally employed as a solvent or suspending medium. For this purpose any bland fixed oil may be employed, including synthetic mono-or diglycerides. In addition, fatty acids such as oleic acid find use in the preparation of injectables.

Pharmaceutically acceptable carriers are formulated according to a number of factors well within the purview of those of ordinary skill in the art. These include, without limitation: the type and nature of the active agent being formulated: the subject to which the agent-containing composition is to be administered: the intended route of administration of the composition: and the therapeutic indication being targeted. Pharmaceutically acceptable carriers include both aqueous and non-aqueous liquid media, as well as a variety of solid and semi-solid dosage forms. Such carriers can include a number of different ingredients and additives in addition to the active agent, such additional ingredients being included in the formulation for a variety of reasons, e.g., stabilization of the active agent, binders, etc., well known to those of ordinary skill in the art. Descriptions of suitable pharmaceutically acceptable carriers, and factors involved in their selection, are found in a variety of readily available sources such as, for example, Allen, L. V. Jr. et al. Remington: The Science and Practice of Pharmacy (2 Volumes), 22nd Edition (2012), Pharmaceutical Press.

Pharmaceutical compositions of this invention comprise at least one compound of Formula (Ib) and optionally an additional agent selected from any pharmaceutically acceptable carrier, adjuvant, and vehicle. Alternate compositions of this invention comprise a compound of the Formula (Ib) described herein, or a prodrug thereof, and a pharmaceutically acceptable carrier, adjuvant, or vehicle.

The selected dosage level will depend upon a variety of factors including the activity of the particular compound of Formula (Ib) employed, the route of administration, the time of administration, the rate of excretion or metabolism of the particular compound being employed, the rate and extent of absorption, the duration of the treatment, other drugs, compounds and/or materials used in combination with the particular compound employed, the age, sex, weight, condition, general health and prior medical history of the patient being treated, and like factors well known in the medical arts.

The above other therapeutic agents, when employed in combination with the compounds of Formula (Ib), may be used, for example, in those amounts indicated in the Physicians' Desk Reference (PDR) or as otherwise determined by one of ordinary skill in the art. In the methods of the present invention, such other therapeutic agent(s) may be administered prior to, simultaneously with, or following the administration of the inventive compounds.

### LIST OF ABBREVIATIONS

Bp Binding potential
BTK Burtons Tyrosine Kinase
[¹¹C]CO₂ Carbon-11 labelled carbon dioxide
[¹¹C]CO Carbon-11 labelled carbon monoxide
CFA complete Freund's Adjuvant
CT computed tomography
DMF dimethyl formamide
EAE Experimental autoimmune encephalomyelitis
FBP filtered back projection
FOV field of view
GBq gigabecquerel
h hour(s)
HPLC High pressure liquid chromatography
HBTU 2-(1H-benzotriazol-1-yl)-1,1,3,3-tetramethyluronium hexafluorophosphate
IC₅₀ half maximal inhibitory concentration
ID injected dose
IV intravenous
MAP maximum a posteriori
MBq megabecquerel
MOA mechanism of action
MRI magnetic resonance imaging
mCi millicurie
mL milliliter
mm millimeter
min minute(s)
NHP nonhuman primate
nM nanomolar
Nm nanometer
Pd(dba)₂ Palladium(0) bis (dibenzylideneacetone)
PET positron emission tomography
PLP Proteolipid protein
ROI region of interest
S.C. subcutaneous
SJL Swiss Jim Lambert
SPE Solid phase extraction
SUV standardized uptake value
SD standard deviation
t ½ time of half-life
TAC time-activity curve
THF tetrahydrofuran
TFA trifluoroacetic acid
Vnd non-specific volume of distribution
Vt volume of distribution
WT wild type
μCi microcurie
μL microliter
μm micrometer

### EXAMPLES

### HPLC Conditions

Method A: GE FXCPro HPLC and GE FXCPro gamma ram radio-HPLC detector using the following method: Column: Luna C18(2), 9.6×250 mm, 5-μm particles; Mobile Phase: Isocratic: 51% acetonitrile in aqueous 0.1% trifluoroacetic acid; Flow: 4.2 mL/min; Detection: UV at 240 nm.

Method B: Agilent 1100 series HPLC and Lab logic gamma ram radio-HPLC detector using the following method Column: Luna C18(2)—250×4.6 mm—3-μm particles analytical HPLC column; mobile phase A: 0.1% aqueous TFA B: 0.1% TFA in acetonitrile. Gradient method consisting of a solution starting at 5% B and increased to 85% B over a 15 minute linear gradient; Flow: 1.00 mL/min; Detection: UV at 254 nm.

Method C: Agilent 1100 series HPLC and Lab logic gamma ram radio-HPLC detector using the following method Column: Luna C18(2)—250×4.6 mm—3-μm particles analytical HPLC column; mobile phase Isocratic: 51% acetonitrile in aqueous 0.1% trifluoroacetic acid; Flow: 1.0 mL/min; Detection: UV at 254 nm.

### Synthesis Of Compound (IB)

(R)-5-fluoro-2,3-dimethyl-4-(1,2,3,4-tetrahydroisoquinolin-5-yl)-1H-indole-7-carboxamide can be prepared according to the process disclosed for Intermediate 106 in WO 2016/065226.

### Example 1

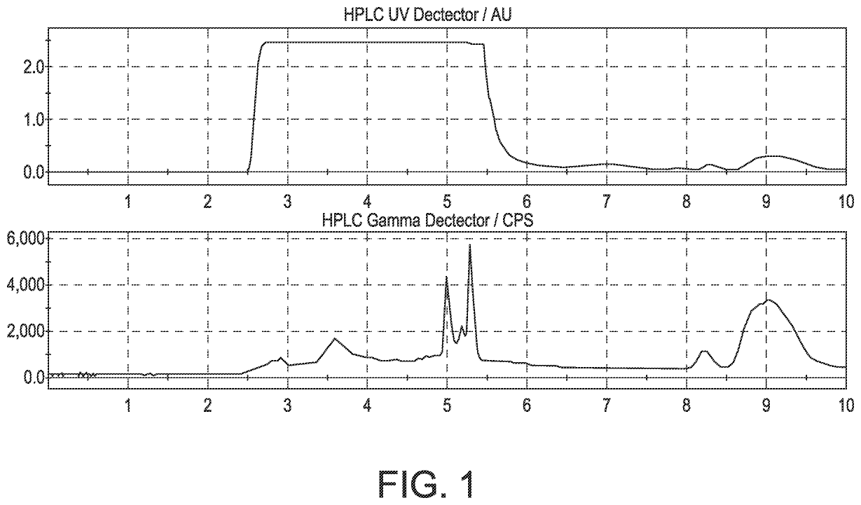

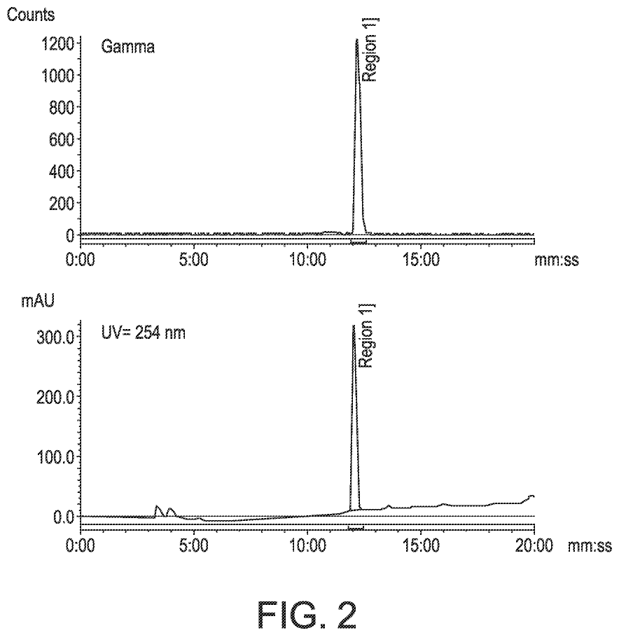

Preparation of [¹¹C]-(R)-4-(2-acryloyl-1,2,3,4-tetrahydroisoquinolin-5-yl)-5-fluoro-2,3-dimethyl-1H-indole-7-carboxamide by Process A

[¹¹C]CO₂ was produced by a nuclear reaction ¹⁴N (P,α)¹¹C using a mixture of nitrogen (N60 purity grade) and 1% oxygen using a high performance [¹¹C]CO₂ target and a GE PET trace cyclotron. The [¹¹C]CO₂ gas was transferred to a molecular sieves column at ambient temperature using a steady stream of helium gas. Upon completion of target delivery, the [¹¹C]CO₂ gas was released from the molecular sieves column by heating the column at 350° C. and helium gas at a flow rate of 7 mL/minute. The [¹¹C]CO₂ was trapped into a vial containing vinyl magnesium bromide (80 μL, 0.056 mmol) and tetrahydrofuran (THF) (320 μL) at 5° C. Upon completion of the [¹¹C]CO₂ transfer, the reaction mixture was warmed to 25° C. and stirred for 2 minutes. The reaction mixture was transferred to an intermediate vial containing 2% trifluoroacetic acid (TFA) in 250 μL of dimethylformamide (DMF). This solution was stirred at ambient temperature for 1 minute. An additional 200 μL of DMF and 50 mL of 2,6-lutidine was transferred to this vial. The reaction mixture was stirred. Next, the entire volume of this solution was transferred to a new intermediate vial containing 100 μL of 2,6-lutidine. After the transfer was completed, 2-(1H-benzo [d] [1,2,3]triazol-1-yl)-1,1,3,3-tetramethylisouronium hexafluorophosphate(V) (5 mg, 0.013 mmol) (HBTU) in 200 μL of DMF was added. The reaction mixture was allowed to stir at ambient temperature for one minute. Next, a solution of (R)-5-fluoro-2,3-dimethyl-4-(1,2,3,4-tetrahydroisoquinolin-5-yl)-1H-indole-7-carboxamide (4 mg, 0.012 mmol) dissolved in 200 μL of DMF and 50 μL of 2,6-lutidine were added to the reaction mixture. The reaction mixture was stirred at ambient temperature for an additional 9.5 minutes. Next, 500 μL of DMF was added to the reaction mixture and the entire sample was transferred to a new intermediate vial containing 8 mL deionized water sample. This sample was loaded onto a C18 (360 mg) solid phase extraction (SPE) cartridge. This cartridge was pre-activated with 5 mL of acetonitrile followed by 10 mL of sterile water for injection before the synthesis. After the entire sample was loaded, the SPE was eluted with 1.4 mL of acetonitrile into a sample containing 0.6 mL of 0.1% TFA in deionized water. The reaction mixture was loaded onto a Luna C18(2), 5 micron, 9.6×250 mm high pressure liquid chromatography (HPLC) column and purified using HPLC Method A purification method. [¹¹C]-(R)-4-(2-acryloyl-1,2,3,4-tetrahydroisoquinolin-5-yl)-5-fluoro-2,3-dimethyl-1H-indole-7-carboxamide was isolated between the 9.5 and 10.5 mark of the chromatogram and the sample was collected into a dilution flask containing 55 mL of a 2 mg/mL sodium ascorbate aqueous solution as shown in FIG. 1. The solution was transferred to a HLB light (30 mg) SPE cartridge. The cartridge was pre-activated with 5 mL of ethanol followed by 10 mL of sterile water before the synthesis. After transfer, the cartridge was eluted with 0.7 mL of ethanol into the sterile product vial containing 4 mL of sterile saline for injection. 28 mCi (1.04 GBq) [¹¹C]-(R)-4-(2-acryloyl-1,2,3,4-tetrahydroisoquinolin-5-yl)-5-fluoro-2,3-dimethyl-1H-indole-7-carboxamide was isolated and analyzed via reverse phase HPLC for as shown in FIG. 2: chemical identify with co-injection of non-radioactive standard, radiochemical purity and chemical purity, molar activity. The isolated product co-eluted with the non-radioactive reference standard. The sample was 99.9% radiochemically pure, 97% chemically pure, with a molar activity of 1 mCi/nmol (38 MBq/nmol). Analytical reverse phase HPLC was used to determine structural identity, radiochemical purity and chemical purity using HPLC method B. The retention time of [¹¹C]-(R)-4-(2-(acryloyl-1)-1,2,3,4-tetrahydroisoquinolin-5-yl)-5-fluoro-2,3-dimethyl-1H-indole-7-carboxamide was 12.03 minutes using this method. Molar activity was determined using a 6-point standard curve (analytical HPLC peak area (Y) versus standard concentration (X: in nmol). The fitted line equation was determined using reverse phase HPLC method C.

The total synthesis time for this process was 40 minutes and the product was isolated in a 3.7% decay corrected yield.

### Example 2

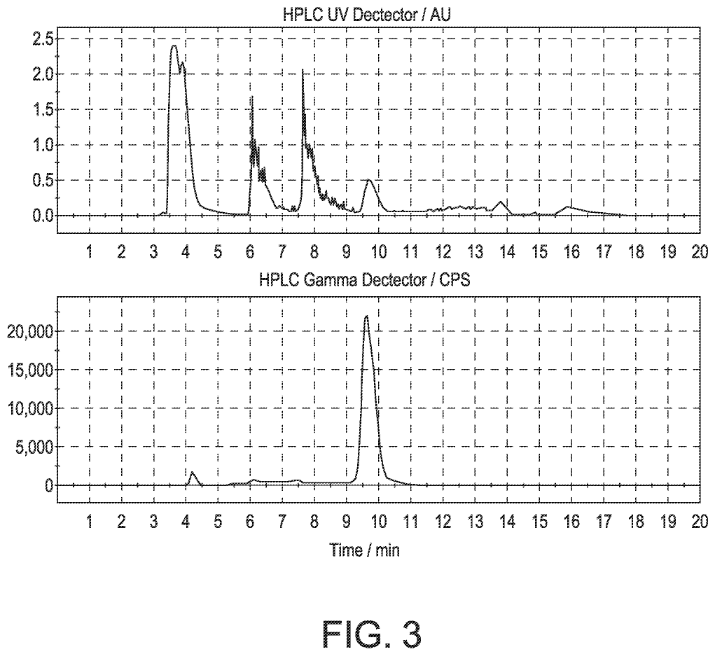

Preparation of [¹¹C]-(R)-4-(2-acryloyl-1,2,3,4-tetrahydroisoquinolin-5-yl)-5-fluoro-2,3-dimethyl-1H-indole-7-carboxamide by Process B

[¹¹C]CO₂ was produced by a nuclear reaction ¹⁴N (P,α)¹¹C using a mixture of nitrogen (N60 purity grade) and 1% oxygen using a high performance [¹¹C]CO₂ target and a GE PET trace cyclotron. The [¹¹C]CO₂ gas was transferred to a molecular sieves column at ambient temperature using a steady stream of helium gas. Upon completion of target delivery, [¹¹C]CO₂ gas was released from the molecular sieves column by heating the column at 350° C. and helium gas at a flow rate of 55 mL/minute and delivered to a quartz tube that contained molybdenum. Once the [¹¹C]CO₂ was trapped onto the molybdenum powder (350 micron particle size 99.9% pure) and was reduced to [¹¹C]CO using a 850° C. furnace. The [¹¹C]CO was delivered from the processing unit at a flow rate of 55 mL/min through a stainless steel trap containing 100 mg of silica gel (230-400 mesh, 60 A) at liquid nitrogen temperatures (−160° C.). After the [¹¹C]CO was trapped within the silica gel trap, the trap was slowly warmed to ambient temperature by removing the liquid nitrogen trap for 2 minutes. Helium gas at 5 mL/minute was used to transfer the [¹¹C]CO into a 5 mL HPLC stainless steel loop containing the following solution. First, bis(dibenzylideneacetone)palladium (0) (2.7 mg, 4.70 μmol) was dissolved in 200 μL of THF and this solution was added to a sample of n-Xantphos 97% (2.7 mg. 4.90 μmol) and the sample was vortexed to dissolve the solids for 1 minute at ambient temperature. To this solution was added vinyl iodide, 95%, 5 g (3 μL, 0.041 mmol) and the resulting solution was vortexed for an additional minute to ensure proper mixing of the precursor materials. To this solution was added to (R)-5-fluoro-2,3-dimethyl-4-(1,2,3,4-tetrahydroisoquinolin-5-yl)-1H-indole-7-carboxamide (1.7 mg, 5.04 μmol) and the crude mixture was vortexed for 30 seconds. This solution was added to the stainless steel HPLC loop before the [¹¹C]CO was delivered to the hot cell. [¹¹C]CO was transferred from the silica trap using helium gas at 5 mL/minute and the transfer time was roughly 20 seconds. The reaction occurred within the sealed 2 mL stainless steel HPLC loop at ambient temperature for 5 minutes. The crude reaction mixture was loaded onto Luna C18(2), 5 micron, 9.6×250 mm semi-prep HPLC column and purified using the following HPLC purification method A. The [¹¹C]-(R)-4-(2-acryloyl-1,2,3,4-tetrahydroisoquinolin-5-yl)-5-fluoro-2,3-dimethyl-1H-indole-7-carboxamide was isolated between the 9.5 and 10.5 mark of the chromatogram and this sample was collected into a dilution flask that contained 55 mL of a 2 mg/mL sodium ascorbate aqueous solution as shown in FIG. 3. This solution was transferred to a HLB light (30 mg) SPE cartridge. This cartridge was pre-activated with 5 mL of ethanol followed by 10 mL of sterile water before the synthesis. After transfer, the cartridge was eluted with 0.7 mL of ethanol into the sterile product vial containing 4 mL of sterile saline for injection. 130 mCi (1.04 GBq) [¹¹C]-(R)-4-(2-acryloyl-1,2,3,4-tetrahydroisoquinolin-5-yl)-5-fluoro-2,3-dimethyl-1H-indole-7-carboxamide was isolated and analyzed via reverse phase HPLC using the following methods. Chemical identify with co-injection of non-radioactive standard, radiochemical purity and chemical purity, molar activity. The isolated product co-eluted with non-radioactive reference standard. The sample was 99.9% radiochemically pure, 97% chemically pure, with a molar activity of 13 mCi/nmol (481 MBq/nmol). Analytical reverse phase HPLC method B was used to determine structural identity, radiochemical purity and chemical purity. The retention time of the [¹¹C]-(R)-4-(2-(acryloyl-1)-1,2,3,4-tetrahydroisoquinolin-5-yl)-5-fluoro-2,3-dimethyl-1H-indole-7-carboxamide was 12.03minutes using this method. Molar activity was determined using a 6-point standard curve (analytical HPLC peak area (Y) versus standard concentration (X: in nmol). The fitted line equation was determined using reverse phase HPLC method C.

The synthesis and purification time was approximately 33 minutes from the end of cyclotron bombardment and the product was isolated in a 42.3% decay corrected yield.

The [¹¹C]-(R)-4-(2-acryloyl-1,2,3,4-tetrahydroisoquinolin-5-yl)-5-fluoro-2,3-dimethyl-1H-indole-7-carboxamide can be used in a variety of in vitro and/or in vivo imaging applications, including diagnostic imaging, basic research, and radiotherapeutic applications. Specific examples of possible diagnostic imaging and radiotherapeutic applications, include determining the location, the relative activity and/or quantifying BTK positive tissues, radioimmunoassay of BTK positive tissues, and autoradiography to determine the distribution of BTK positive tissues in a mammal or an organ or tissue sample thereof. In particular, [¹¹C]-(R)-4-(2-acryloyl-1,2,3,4-tetrahydroisoquinolin-5-yl)-5-fluoro-2,3-dimethyl-1H-indole-7-carboxamide is useful for positron emission tomographic (PET) imaging of BTK positive tissues within in the spleen, brain, heart, kidneys, liver and skin and other organs of humans and experimental animals. PET imaging using the [¹¹C]-(R)-4-(2-acryloyl-1,2,3,4-tetrahydroisoquinolin-5-yl)-5-fluoro-2,3-dimethyl-1H-indole-7-carboxamide can be used to obtain the following information: relationship between level of tissue occupancy by BTK inhibitor candidate in medicaments and clinical efficacy in patients; dose selection for clinical trials of BTK inhibitor treating medicaments prior to initiation of long term clinical studies; comparative potencies of structurally novel BTK inhibitor treating medicaments; investigating the influence of BTK inhibitor treating medicaments on in vivo transporter affinity and density during the treatment of clinical targets with BTK inhibitors treating medicaments; changes in the density and distribution of BTK positive tissues during effective and ineffective treatment.

[¹¹C]-(R)-4-(2-acryloyl-1,2,3,4-tetrahydroisoquinolin-5-yl)-5-fluoro-2,3-dimethyl-1H-indole-7-carboxamide was tested to confirm its properties as a BTK PET radioligand. [¹¹C]-(R)-4-(2-acryloyl-1,2,3,4-tetrahydroisoquinolin-5-yl)-5-fluoro-2,3-dimethyl-1H-indole-7-carboxamide was tested for its specificity and targeting of BTK within the brain tissues of wild type mice and mice in a PLP induced relapsing/remitting EAE mouse model. The EAE induced relapsing/remitting model was induced using a series of Swiss Jim Lambert (SJL) mice were immunized with proteolipid protein (PLP). To induce relapsing/remitting EAE mouse model, SJL/J female mice (Harlan, 9-11 weeks/˜20 grams) were injected with 50 μg of mouse PLP 139-151 in complete Freund's Adjuvant (CFA) containing 2 mg/mL Mycobacterium tuberculosis H37Ra (Difco, Detroit, MI) at 2 sites (0.1 ml/site) on the back (between shoulder blades and above the right hip). Two hours later, mice were injected intraperitoneally with 100 μL containing 300 ng Pertussis Toxin. Two days later the mice were injected with an additional intraperitoneally injection of 100 μL of 300 ng Pertussis Toxin. Mice were then returned to their cages and were monitored daily for the duration of the experiment. The PET imaging was acquired between days 40-45 following EAE induction. Mice (EAE and WT) were kept immobilized in an animal holder inside the gantry of the PET imaging system (MicroPET® P4, Siemens Preclinical Solutions, Knoxville, TN). The scanning region was from approximately the nose back toward the tail of the animal, to fit into the 7.6 cm MicroPET system axial field of view (FOV). A 10-minute transmission scan using a ⁵⁷Co source for the purpose of attenuation correction of the emission PET images were performed for the FOV described above, followed by a 90-minute dynamic emission scan in the PET studies. Approximately 232 μCi of [¹¹C]-(R)-4-(2-acryloyl-1,2,3,4-tetrahydroisoquinolin-5-yl)-5-fluoro-2,3-dimethyl-1H-indole-7-carboxamide was injected via tail vein catheter for each animal. Blocking studies were completed in EAE animals by giving either 5 mg/kg or 0.5 mg/kg oral doses to the EAE mice immediately after the baseline imaging study. An additional 5 mg/kg or 0.5 mg/kg oral dose was given to the same EAE mice 24 hours after the baseline study. On-drug PET images were acquired 28 hours after the original scan following a second administration of [¹¹C]-(R)-4-(2-acryloyl-1,2,3,4-tetrahydroisoquinolin-5-yl)-5-fluoro-2,3-dimethyl-1H-indole-7-carboxamide following the same image acquisition procedures. PET images were reconstructed with a maximum a posteriori (MAP) algorithm with attenuation correction and radioisotope decay correction. Analysis for each mouse was done with regions of interest (ROIs) manually drawn within each brain section of both wild type and EAE mouse brain. A single ROI was drawn within the brain of WT mice and 2 ROIs drawn in the brain for each EAE mouse (suspected EAE lesion regions and background brain areas). Standard uptake values were calculated based on these ROIs, and time-activity curves (TACs) were generated to determine tracer uptake over the course of the imaging time points. Using this methodology, a test/retest PET study was performed in EAE mice at days 40-45 post EAE induction. 2 EAE animals received a baseline PET image and a follow up baseline imaging study (PET ligand injection only). Acquisition data for [¹¹C]-(R)-4-(2-acryloyl-1,2,3,4-tetrahydroisoquinolin-5-yl)-5-fluoro-2,3-dimethyl-1H-indole-7-carboxamide is summarized in Table 1:

TABLE 1
Acquisition data for [¹¹C]-(R)-4-(2-acryloyl-
1,2,3,4-tetrahydroisoquinolin-5-yl)-fluoro-2,3-dimethyl-
1H-indole-7-carboxamide PET Imaging in Rodents

| Injected Dose | | |
|---|---|---|
| (mCi) | Weight (g) | Study |
| 0.166 | 19 | EAE |
| 0.258 | 21 | WT |
| 0.130 | 21 | EAE |
| 0.512 | 19 | EAE block |
| 0.175 | 20 | EAE |
| 0.114 | 20 | EAE block |
| 0.203 | 20 | EAE |
| 0.085 | 19 | EAE retest |
| 0.270 | 22 | WT |
| 0.190 | 21 | WT |
| 0.12 | 22 | EAE test |
| 0.14 | 21 | EAE test |
| 0.18 | 23 | EAE retest |
| 0.13 | 19 | EAE retest |
| 0.11 | 24 | EAE baseline |
| 0.0638 | 21 | EAE baseline |
| 0.1211 | 24 | EAE 0.5 mpk block |
| 0.1553 | 24 | EAE baseline |
| 0.1041 | 23 | EAE baseline |
| 0.0798 | 24 | EAE 0.5 mpk block |
| 0.0515 | 23 | EAE 0.5 mpk block |
| 0.0399 | 26 | EAE baseline |
| 0.0562 | 20 | EAE baseline |
| 0.0897 | 26 | EAE 5 mpk block |
| 0.0639 | 20 | EAE 5 mpk block |
| 0.0712 | 21 | EAE baseline |
| 0.044 | 22 | EAE baseline |
| 0.173 | 20 | EAE 5 mpk block |
| 0.131 | 22 | EAE 5 mpk block |

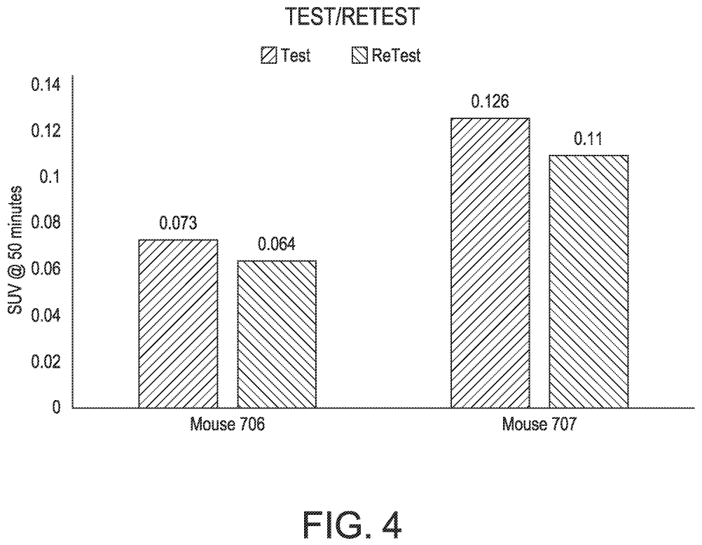

Results for these studies are shown in FIGS. 4-6). As shown in FIG. 4, these results demonstrate similar PET ligand uptake within the EAE lesion region in both obtained PET imaging scans. 0.073 SUV was measured within the EAE lesion on the first baseline scan and that same lesion region was measured to be a 0.064 SUV on the follow up baseline scan. A similar result was seen in another EAE animal, with 0.126 SUV was measured within the EAE lesion upon baseline, with a 0.11 SUV on the follow up baseline scan. These results show a 13% variability between scans for this PET ligand, which is within typical variation seen in PET ligand imaging scans.

FIG. 4: Standard uptake values (SUV) of [¹¹C]-(R)-4-(2-acryloyl-1,2,3,4-tetrahydroisoquinolin-5-yl)-5-fluoro-2,3-dimethyl-1H-indole-7-carboxamide PET signal showing retention of ligand within the EAE lesion region of the mouse brain in 2 EAE induced mice. The blue bars indicate SUV values within the lesion region of the mouse brain 50 minutes after ligand administration. The red bars indicate follow-up SUV values within the lesion region of the mouse brain 50 minutes after ligand administration within the same animals. These follow-up images were acquired 24 hours later after a second administration of the PET ligand. Each set of bars represents data from an individual mouse.

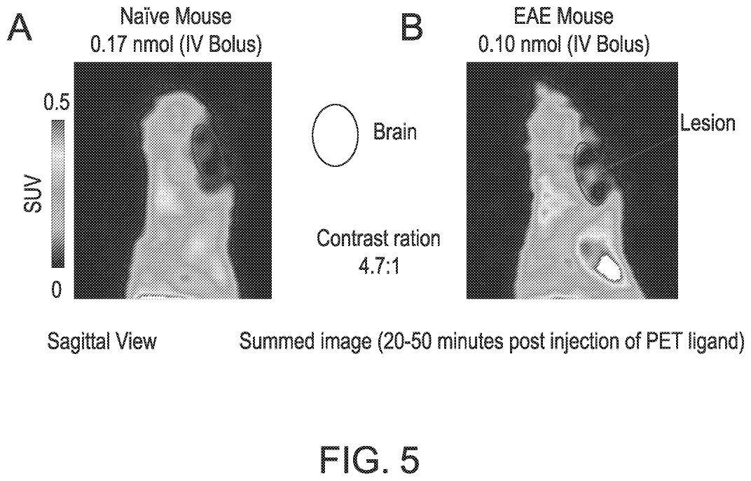

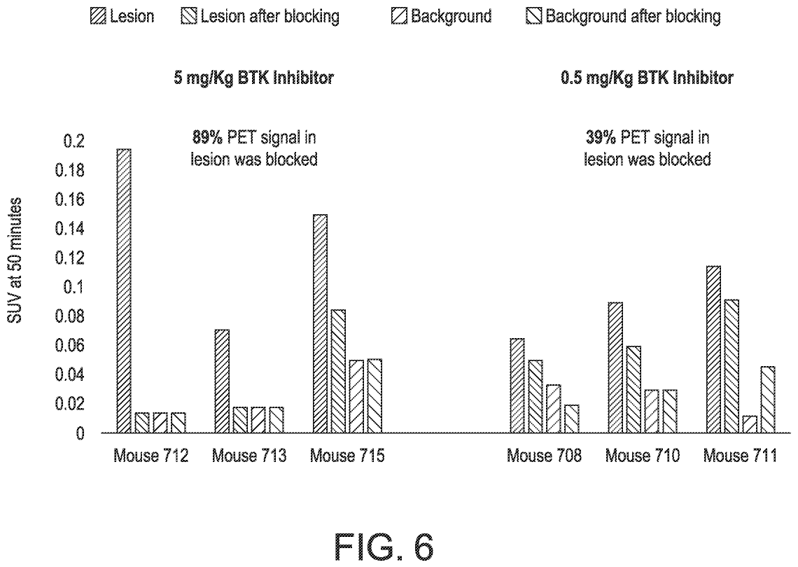

FIG. 5, shows representative PET images of brain accumulation of the PET ligand in WT and EAE mice. In WT mice, there is very low brain expression of BTK in a non-diseased state. [¹¹C]-(R)-4-(2-acryloyl-1,2,3,4-tetrahydroisoquinolin-5-yl)-5-fluoro-2,3-dimethyl-1H-indole-7-carboxamide TACs demonstrated rapid brain penetration followed by fast washout as the drug was cleared. FIG. 5 also highlights this, with only background levels of [¹¹C]-(R)-4-(2-acryloyl-1,2,3,4-tetrahydroisoquinolin-5-yl)-5-fluoro-2,3-dimethyl-1H-indole-7-carboxamide remaining within the brain between the 20-50 minute post radioligand administration. In contrast to non-diseased mice, PET imaging studies in EAE mice showed that after drug washed out of the brain, focal retention of the ligand was evident in an area corresponding to the lateral sides of the medulla oblongata where the inflammatory lesions are typically observed in this EAE animal model. FIG. 5 also demonstrated the focal retention within a lesion area within the EAE mouse brain of [¹¹C]-(R)-4-(2-acryloyl-1,2,3,4-tetrahydroisoquinolin-5-yl)-5-fluoro-2,3-dimethyl-1H-indole-7-carboxamide was measured to have an SUV of 0.141compared to the area outside the brain having a SUV of 0.03, resulting in a contrast ratio of 4.7:1. This result suggests that these lesion areas within brain of an EAE mouse can be visualized using PET imaging. To assess BTK inhibitor drug target engagement and dose-dependent displacement of BTK PET signal in EAE lesions of mouse brains, EAE induced animals received a baseline (PET ligand only) scan with [¹¹C]-(R)-4-(2-acryloyl-1,2,3,4-tetrahydroisoquinolin-5-yl)-5-fluoro-2,3-dimethyl-1H-indole-7-carboxamide. Next, a second [¹¹C]-(R)-4-(2-acryloyl-1,2,3,4-tetrahydroisoquinolin-5-yl)-5-fluoro-2,3-dimethyl-1H-indole-7-carboxamide administration and serial PET scan was obtained in the same animals following oral dosing with two doses of a BTK inhibitor (either 5 mg/kg or 0.5 mg/kg, n=3 for each dose group). Oral dosing occurred at 28 hours and again at 4 hours prior to the second tracer injection. FIG. 6, shows the results of these 2 separate dose panels for target engagement. In the first dosing panel, animals were scanned at baseline and showed an average SUV of 0.14 within the lesion area of these mice and an SUV of 0.039 outside that region. After 2 oral doses of a BTK inhibitor at 5 mg/kg, the average SUV within the lesion region was reduced to 0.05, while intensity outside of the suspected region remained largely unchanged. The average percent displacement of this dose panel was 89% in suspected lesions. A similar study was completed at the 0.5 mg/kg dose panel which resulted in an SUV of 0.09 within the lesion area of the EAE mice. After blocking at the 0.5 mg/kg dosing level, the average percent displacement was 39% in suspected lesions. These results suggest that dose dependent target engagement of BTK inhibitor can be measured with this ¹¹C-labeled BTK PET ligand within brain lesions of EAE induced mice.

FIG. 6: Representative bar graphs from quantification of the PET images in EAE induced mouse brains. 2 ROIs were defined within this study, the lesion area and an area of the brain outside of lesions, showing brain accumulation of [¹¹C]-(R)-4-(2-acryloyl-1,2,3,4-tetrahydroisoquinolin-5-yl)-5-fluoro-2,3-dimethyl-1H-indole-7-carboxamide (SUV from 50 minutes following PET ligand administration). The red bar represents the lesion area within the brain. The blue bar represents the lesion area during the dosing panel (either 5 or 0.5 mg/kg BTK inhibitor. The green bar represents a representative ROI within the mouse brain, outside of the lesion region at baseline. The yellow bar represents a representative ROI within the mouse brain, outside of the lesion region during dosing of BTK inhibitor.

### Example 5

In-Vivo PET Imaging With [¹¹C]-(R)-4-(2-acryloyl-1,2,3,4-tetrahydroisoquinolin-5-yl)-5-fluoro-2,3-dimethyl-1H-indole-7-carboxamide in Rhesus Monkey to Determine Brain Penetrance of BTK Inhibitors.

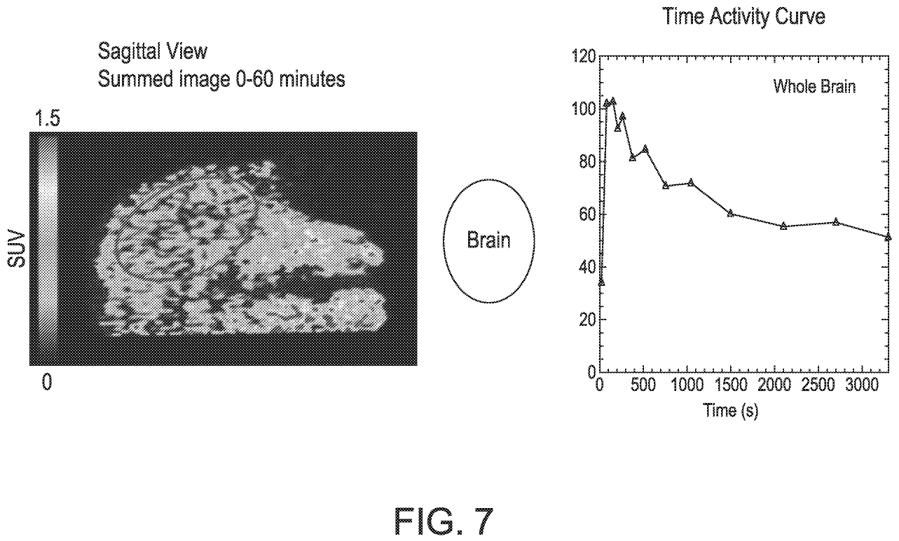

The non-human primate was kept immobilized in the extended imaging bed inside the gantry of the PET imaging system (MicroPET® P4, Siemens Preclinical Solutions, Knoxville, TN). The scanning region was a single bed position from approximately the top of skull and extending through the lower neck area, to fit into the 7.6 cm MicroPET system axial FOV. A 10-minute transmission scan was acquired using a ⁵⁷Co point source for the purpose of attenuation correction of the subsequent emission image. Approximately 0.96 mCi of [¹¹C]-(R)-4-(2-acryloyl-1,2,3,4-tetrahydroisoquinolin-5-yl)-5-fluoro-2,3-dimethyl-1H-indole-7-carboxamide was injected IV and a 60-minute dynamic PET image was acquired. PET images were reconstructed with a filtered back projection (FBP) or MAP algorithm with attenuation correction. Using the AMIDE software package, an ROI encompassing the whole brain was manually drawn in the PET images. Mean standardized uptake value (SUV), which is normalized with body weight (kg) and injected dose (mCi), was calculated and plotted as a function of time to obtain a TAC. This baseline imaging study (PET ligand only) in a healthy rhesus monkey confirmed brain penetrance within the non-human primate brain. Diffuse uptake was observed throughout the brain of this monkey as shown in FIG. 7. Summed images from 0-60 minutes post injection of the PET ligand demonstrate this within the NHP. In healthy rhesus monkey, as in WT mice. there is very low brain expression of BTK in a non-diseased state. [¹¹C]-(R)-4-(2-acryloyl-1,2,3,4-tetrahydroisoquinolin-5-yl)-5-fluoro-2,3-dimethyl-1H-indole-7-carboxamide TACs showed a rapid brain penetration followed by washout as the ligand was cleared.

What is claimed is:

1. A radiolabeled compound of Formula (Ib):

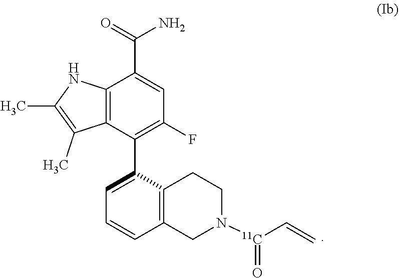

2. A composition comprising compound of Formula (Ia) and compound of Formula (Ib): 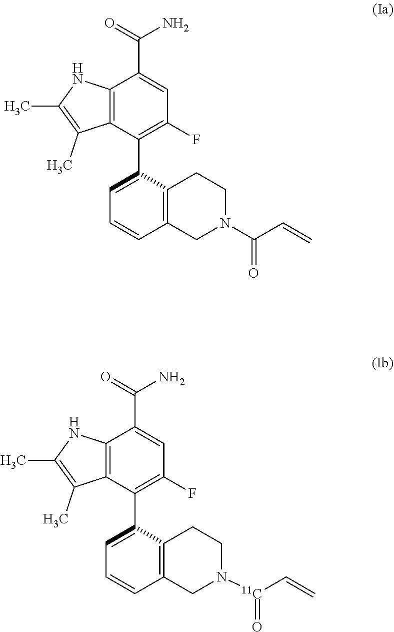
having from 10 to 100 mole % compound of Formula (Ib) based on the moles of compound of Formula (Ia) and compound of Formula (Ib).

3. A pharmaceutical composition comprising the radiolabeled compound of Formula (Ib) according to claim 1; and a pharmaceutically acceptable carrier.

4. The pharmaceutical composition comprising 5 to 25 millicuries of the compound of Formula (Ib) and a pharmaceutically acceptable carrier.

5. The pharmaceutical composition according to claim 3 wherein said pharmaceutically acceptable carrier is saline solution.

6. The pharmaceutical composition according to claim 5, further comprising ethanol.

7. A method of in vivo imaging of mammalian tissues of known BTK expression comprising the steps of:
(a) administering the radiolabeled compound of Formula (Ib) according to claim 1 to a mammalian species; and
(b) imaging in vivo the distribution of the radiolabeled compound by positron emission tomography (PET) scanning.

6. (canceled)

7. (canceled)

8. A method for screening a non-radiolabeled compound to determine its affinity for occupying the binding sites of BTKs in mammalian tissue comprising the steps of:
(a) administering the radiolabeled compound of Formula (Ib) according to claim 1 to a mammalian species;
(b) imaging in vivo tissues of known BTK expression by positron emission tomography (PET) to determine a baseline uptake of the radiolabeled compound;
(c) administering the non-radiolabeled compound to the mammalian species;
(d) administering a second dose of the radiolabeled compound to the mammalian species;
(e) imaging in vivo the distribution of the radiolabeled compound in tissues that express BTK;
(f) comparing the signal from PET scan data at the baseline within the tissue that expresses BTK to PET scan data retrieved after administering the non-radiolabeled compound within the tissue that expresses BTK receptors.

9. A method for monitoring the treatment of a mammalian patient who is being treated with an BTK inhibitor comprising the steps of:
(a) administering to the patient the radiolabeled compound of Formula (Ib) according to claim 1;
(b) obtaining an image of tissues in the patient that express BTK by positron emission tomography (PET); and
(c) detecting to what degree the radiolabeled compound occupies the binding site of the BTK.

10. A method for tissue imaging comprising the steps of:
(a) contacting a tissue that contains BTK with the radiolabeled compound of Formula (Ib) according to claim 1; and
(b) detecting the radiolabeled compound using positron emission tomography (PET) imaging.

11. A method for diagnosing the presence of a multiple sclerosis in a mammalian species, comprising the steps of:
(a) administering to a mammalian species in need thereof the radiolabeled compound of Formula (Ib) according to claim 1, which binds to the BTK associated with the presence of the multiple sclerosis disease; and
(b) obtaining a radio-image of at least a portion of the mammalian species to detect the presence or absence of the radiolabeled compound;
wherein the presence and location of the radiolabeled compound above background is indicative of the presence or absence of the disease.

12. A method of preparing a compound of Formula (Ib): 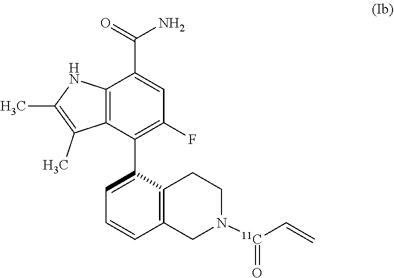
comprising the steps of:
(a) reacting [¹¹C]CO₂ and vinyl magnesium bromide to provide [¹¹C]-acrylic acid having the structure of Formula (II): 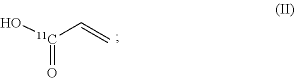
and
(b) reacting said [¹¹C]-acrylic acid and (R)-5-fluoro-2,3-dimethyl-4-(1,2,3,4-tetrahydroisoquinolin-5-yl)-1H-indole-7-carboxamide to provide the compound of Formula (Ib).

13. A method of preparing a compound of Formula (Ib): 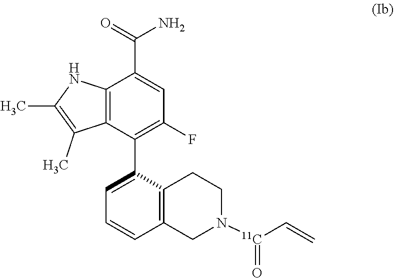
comprising the step of reacting [¹¹C]CO and vinyl iodide in the presence of (R)-5-fluoro-2,3-dimethyl-4-(1,2,3,4-tetrahydroisoquinolin-5-yl)-1H-indole-7-carboxamide to provide the compound of Formula (Ib).

14. A method for diagnosing the presence of a multiple sclerosis in a mammalian species, comprising the steps of:
(a) administering to a mammalian species in need thereof the radiolabeled compound of Formula (Ib) according to claim 1, which binds to the BTK associated with the presence of the multiple sclerosis disease; and
(b) obtaining a radio-image of at least a portion of the mammalian species to detect the presence or absence of the radiolabeled compound;
wherein the presence and location of the radiolabeled compound above background is indicative of the presence or absence of the disease.

15. A method for diagnosing the presence of a multiple sclerosis lesion in the brain of a mammalian species, comprising the steps of:
(a) administering to a mammalian species in need thereof the radiolabeled compound of Formula (Ib) according to claim 1, which binds to the BTK associated with the multiple sclerosis lesion; and
(b) obtaining a radio-image of at least a portion of the brain of the mammalian species to detect the presence or absence of the radiolabeled compound;
wherein the presence and location of the radiolabeled compound above background is indicative of the presence or absence of the multiple sclerosis lesion.
